(19) United States
(12) Patent Application Publication
(10) Pub. No.: US 2017/0294595 A1
(43) Pub. Date: Oct. 12, 2017

(54) HOLE TRANSPORTING MATERIAL FOR AUTOMOTIVE PEROVSKITE SOLAR CELL HAVING HIGH HEAT RESISTANCE, PEROVSKITE SOLAR CELL INCLUDING THE SAME, AND METHOD FOR MANUFACTURING THE SAME

(71) Applicants: HYUNDAI MOTOR COMPANY, Seoul (KR); Korea Research Institute of Chemical Technology, Daejeon (KR)

(72) Inventors: Sol KIM, Suwon-si (KR); Mi Yeon SONG, Seoul (KR); Eun Yeong LEE, Seoul (KR); Sang Hak KIM, Seoul (KR); Moon Jung EO, Suwon-si (KR); Sang IL SEOK, Daejon (KR); Jun Hong NOH, Daejeon (KR); Jang Won SEO, Seoul (KR); Young Chan KIM, Daejeon (KR)

(73) Assignees: HYUNDAI MOTOR COMPANY, Seoul (KR); Korea Research Institute of Chemical Technology, Daejeon (KR)

(21) Appl. No.: 15/481,239

(22) Filed: Apr. 6, 2017

(30) Foreign Application Priority Data
Apr. 6, 2016 (KR) 10-2016-0042355

(51) Int. Cl. H01L 51/00; C07F 1/08

(52) U.S. Cl. CPC H01L 51/0078; C07F 1/08; H01L 51/424

(57) ABSTRACT

A hole transporting material having excellent heat resistance and durability, a perovskite solar cell including the hole transporting material in a hole transporting layer, and a method for manufacturing the solar cell are provided. Provided is a perovskite solar cell having PCE which is equal to or greater than PCE in the related art because the hole transporting layer is formed by using the hole transporting material in which the phthalocyanine-based organic ligand is coordinate-bonded to metal. Also, provided is a perovskite solar cell which can maintain initial PCE for a long time in a wide temperature range when the hole transporting material is used as the hole transporting layer due to excellent heat resistance and durability.

### CROSS-REFERENCE TO RELATED APPLICATION

This application claims under 35 U.S.C. §119(a) the benefit of Korean Patent Application No. 10-2016-0042355 filed on Apr. 6, 2016, the entire contents of which are incorporated herein by reference.

### BACKGROUND

### (a) Field

The present disclosure relates to a hole transporting material having excellent heat resistance and durability, a perovskite solar cell including the hole transporting material as a hole transporting layer, and a method for manufacturing the solar cell.

### (b) Background Art

A perovskite solar cell means a solid-state solar cell based on a light absorbing material having a perovskite (ABX₃) structure.

The perovskite solar cell has a very high absorption coefficient to effectively absorb solar light even with a thickness of submicrometers and thus recently, has received large attention due to good efficiency (about 20% of power conversion efficiency (PCE)).

As a part thereof, in Korean Patent No. 10-1543438, power conversion efficiency is improved by adding a conductive filler such as carbon nanotube to a hole transporting layer of a perovskite solar cell.

In Korean Patent No. 10-1578875, electrons smoothly move by adopting the hole blocking layer to the perovskite solar cell.

The disclosure of this section is to provide background of the invention. Applicant notes that this section may contain information available before this application. However, by providing this section, Applicant does not admit that any information contained in this section constitutes prior art.

### SUMMARY

An aspect of the present invention is to provide a hole transporting material having excellent heat resistance and durability as a material which can be applied to a hole transporting layer of a perovskite solar cell.

Another aspect of the present invention is to provide a hole transporting material which can form a hole transporting layer by a solution casting process, not an expensive process such as deposition.

One aspect of the present invention provides a hole transporting material for a perovskite solar cell having high heat resistance in which a phthalocyanine-based organic ligand is coordinate-bonded to metal.

In an embodiment, the metal may be copper (Cu), zinc (Zn) or cobalt (Co).

In another embodiment, the phthalocyanine-based organic ligand may include a tert-butyl substituent.

In still another embodiment, the hole transporting material for the perovskite solar cell having high heat resistance may be expressed by Chemical Formula 1 below.

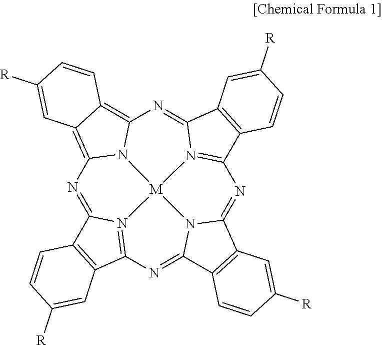

Herein, the M is copper (Cu) and the R is tert-butyl.

Another aspect of the present invention provides a perovskite solar cell including a first electrode, an electron transporting layer formed on the first electrode, a light absorbing layer formed on the electron transporting layer and including a compound having a perovskite structure, a hole transporting layer formed on the light absorbing layer, and a second electrode formed on the hole transporting layer.

In an embodiment, the hole transporting layer may be made of the hole transporting material.

A further aspect of the present invention provides a method for manufacturing a perovskite solar cell including forming a hole transporting layer by solution-casting the hole transporting material.

In an embodiment, the solution-casting may be performed by any one process selected from spin coating, spray coating, slot die, inkjet coating and gravure coating.

According to embodiments of the present invention, the hole transporting material has excellent heat resistance and durability to maintain stability even though the hole transporting material is exposed to a vehicle component integrated packaging process at 100° C. or greater and a vehicle traveling environment.

Unique characteristics of the hole transporting material such as power conversion efficiency do not largely deteriorate even at a high temperature to maintain high efficiency of a perovskite solar cell in a wide temperature range.

Therefore, the perovskite solar cell including the hole transporting material according to embodiments of the present invention as a hole transporting layer is suitable to be applied to a vehicle.

When the hole transporting material according to embodiments of the present invention is used, the hole transporting layer may be formed by a cheap solution casting process to enhance market competitiveness.

A further aspect of the invention provides a method of making a vehicle, the method comprises: providing a vehicular surface; and attaching a film comprising a solar cell which comprises a perovskite layer and a hole transporting layer, wherein the hole transporting layer does not comprise any one of spiro-OMeTAD and PTAA and comprises a composition which comprises a phthalocyanine-based organic ligand coordinate-bonded to a metal such that no phase transition in the hole transporting layer occurs when attaching the film at a temperature above 110° C.

The effects of the present invention are not limited to the aforementioned effects. It should be understood that the effects of the present invention include all effects inferable from the description below.

Other aspects and embodiments of the invention are discussed infra.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The above and other features of embodiments of the invention are discussed infra.

### BRIEF DESCRIPTION OF THE DRAWINGS

The above and other features of the present invention will now be described in detail with reference to certain embodiments thereof illustrated in the accompanying drawings which are given hereinbelow by way of illustration only, and thus are not limitative of the present invention, and wherein:

FIG. 1 schematically illustrates a structure of a perovskite solar cell according to embodiments of the present invention;

FIG. 2 is a result of evaluating heat resistance of a hole transporting material according to Example 1 of the present invention and a result of measuring a heat flow when the hole transporting material is exposed at a temperature of 0° C. to 300° C.;

FIG. 3 is a result of evaluating heat resistance of a hole transporting material according to Example 1 of the present invention and an X-ray diffraction (XRD) result before and after the hole transporting material is heated for 30 minutes at 130° C.;

FIG. 4A is a scanning electron microscope (SEM) photograph of a cross section of the entire perovskite solar cells according to Example 2 of the present invention;

FIG. 4B is scanning electron microscope (SEM) photographs of cross sections of an enlarged light absorbing layer and a hole transporting layer of the perovskite solar cell according to Example 2 of the present invention;

FIG. 5 is a result of measuring power conversion efficiency according to a temperature in a perovskite solar cell according to Example 3 of the present invention and each Comparative Example;

FIG. 6 is a result of evaluating durability of a perovskite solar cell according to Example 4 of the present invention; and

FIG. 7 is a result of measuring current density of a perovskite solar cell according to Example 5 of the present invention and other perovskite solar cells including a high heat resistant hole transporting material.

Reference numerals set forth in the Drawings includes reference to the following elements as further discussed below.

10: first electrode
20: electron transporting layer
30: light absorbing layer
40: hole transporting layer
50: second electrode

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawings.

### DETAILED DESCRIPTION

Hereinafter reference will now be made in detail to various embodiments of the present invention, examples of which are illustrated in the accompanying drawings and described below. While the invention will be described in conjunction with embodiments, it will be understood that present description is not intended to limit the invention to those embodiments. On the contrary, the invention is intended to cover not only the embodiments, but also various alternatives, modifications, equivalents and other embodiments, which may be included within the spirit and scope of the invention as defined by the appended claims.

Hereinafter, the present invention will be described in more detail through embodiments. The embodiments of the present invention may be modified in various forms as long as the gist of the invention is not changed. However, the scope of the present invention is not limited to the following embodiments.

When it is determined that the present invention may obscure the gist of the present invention, the description for the known configurations and functions will be omitted.

In this specification, the term “comprise” means that other constituent elements may be further included unless otherwise listed.

A typical perovskite solar cell uses spiro-OMeTAD [(2,2′, 7, 7′-tetrakis(N, N-di-p-methoxyphenylam ine)9,9′-spirobifluorine)], PTAA [poly(triarylamine)], and the like which have bad heat resistance as the hole transporting material and thus there is a limitation in that it is difficult to be applied to a vehicle.

In order to apply the perovskite solar cell to the vehicle, the perovskite solar cell needs to be integrated with a vehicle component by using an adhesive film, and in this case, a process temperature rises to 110° C. or more. Further, when the vehicle starts traveling, the temperature rises up to about 100° C.

The typical hole transporting material such as spiro-OMeTAD and PTAA may not be applied to the perovskite solar cell for the vehicle because a phase transition (thermal transition) is generated at about 90 to 120° C. and thus characteristics such as the power conversion efficiency are rapidly reduced.

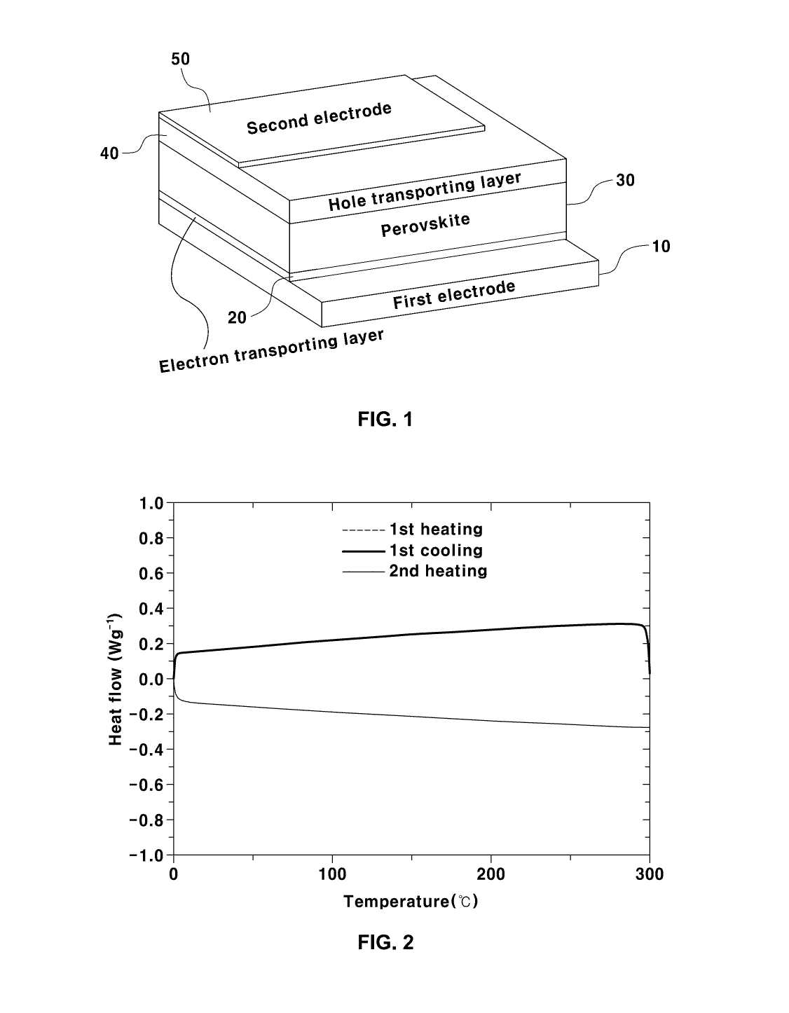

As illustrated in FIG. 1, a perovskite solar cell according to embodiments of the present invention may include a first electrode 10, an electron transporting layer 20 formed on the first electrode 10, a light absorbing layer 30 including a compound having a perovskite structure formed on the electron transporting layer 20, a hole transporting layer 40 formed on the light absorbing layer 30, and a second electrode 50 formed on the hole transporting layer 40.

The electron transporting layer 20 may be formed by any configuration and form so long as electrons smoothly move, but may be formed in a porous layer constituted by metal oxide particles such as titanium dioxide (TiO₂).

The light absorbing layer 30 may be constituted by a light absorption material capable of being expressed by the following Chemical Formula.

ABX₃

Herein, the A may be formamidinium or methylammonium, the B may be lead (Pd), and the X may be iodine (I) or bromine (Br).

Preferably, the light absorption material may use formamidinium lead iodide (FAPbI₃) having good efficiency, but is not limited thereto.

The hole transporting layer 40 may be constituted by a hole transporting material including metal and a phthalocyanine-based organic ligand.

The hole transporting material may be a compound in which metal is positioned at the center and the phthalocyanine-based organic ligand is coordinate-bonded to the periphery of the metal. The metal may use copper (Cu).

The phthalocyanine-based organic ligand may be phthalocyanine or phthalocyanine including a tert-butyl substituent.

Preferably, as the phthalocyanine-based organic ligand, tert-butyl substituted phthalocyanine may be used. The reason is that the phthalocyanine has low solubility for an organic solvent (toluene, chlorobenzene, dichlorobenzene, chloroform, etc.) to form the hole transporting layer 40 only through a vacuum deposition method, whereas the phthalocyanine including the tert-butyl substituent has high solubility for the organic solvent to form the hole transporting layer 40 by a solution casting process.

In an embodiment of the present invention, the hole transporting material may use a compound expressed by the following Chemical Formula 1.

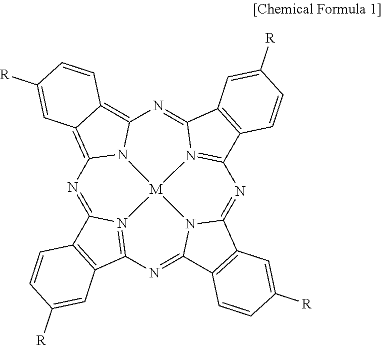

Herein, the M may be copper (Cu) and the R may be tert-butyl.

In embodiments, a method of installing solar cells on a vehicle is provided. The method includes (1) providing a vehicular surface; and (2) attaching a film including a solar cell. The solar cell includes a perovskite layer and a hole transporting layer. The hole transporting layer does not include any one of spiro-OMeTAD and PTAA. Rather, the hole transporting layer includes a composition which includes a phthalocyanine-based organic ligand coordinate-bonded to a metal such that no phase transition in the hole transporting layer occurs when attaching the film to the vehicular surface at a temperature above 110° C.

Hereinafter, the present invention will be described in more detail through Examples. However, these Examples are to exemplify the present invention and the scope of the present invention is not limited thereto.

### EXAMPLES

The following examples illustrate the invention and are not intended to limit the same.

### Example 1

### Evaluation of Heat Resistance of “Hole Transporting Material”

According to an embodiment of the present invention, a hole transporting material expressed by the following Chemical Formula 2 was prepared.

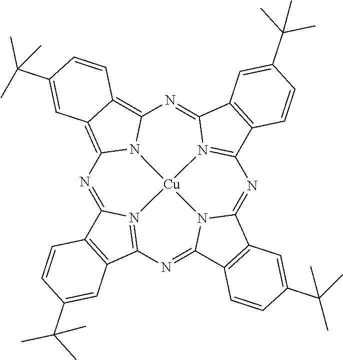

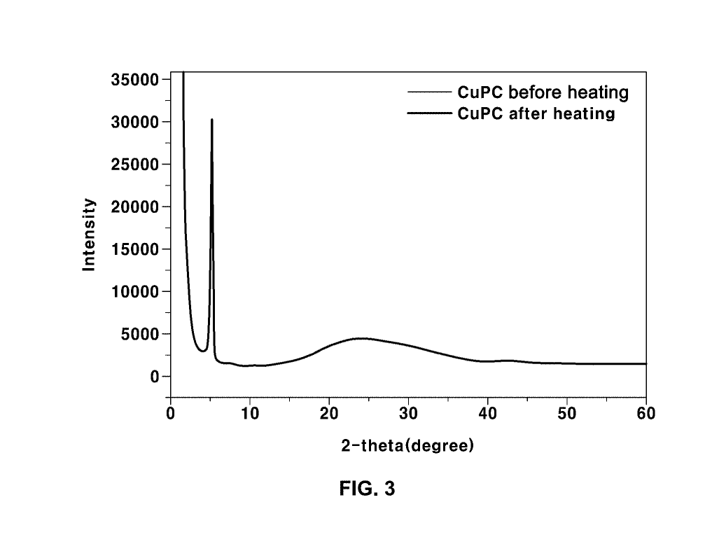

The heat resistance of the hole transporting material was evaluated by a differential scanning calorimetry and an XRD measuring method of a film before and after heating a temperature. The results were illustrated in FIGS. 2 and 3, respectively.

Referring to FIG. 2, it can be seen that a change in heat flow is not large in an average temperature range of 0° C. to 300° C.

When the material is physically (melting, vaporization, etc.) or chemically changed, an exothermic or endothermic phenomenon occurs, and as illustrated in FIG. 2, a case where entry and exit of heat are not large means that a phase transition (thermal transition) of the hole transporting material does not occur within the temperature range.

Referring to FIG. 3, it can be seen that the XRD analysis result before and after the hole transporting material is heated for 30 minutes at 130° C. is not changed.

This means that the crystal structure of the hole transporting material is not changed even after being heated for 30 minutes at 130° C.

As a result, in the hole transporting material according to an embodiment of the present invention, it can be seen that since a phase transition (thermal transition) does not occur in a wide temperature range of 0° C. to 300° C. and a crystal structure is not changed even at a temperature of 130° C. higher than a traveling environment temperature of the vehicle, heat resistance is excellent.

### Example 2

### Preparation of Perovskite Solar Cell

A perovskite solar cell was manufactured by using the hole transporting material of Example 1.

In this case, the first electrode, the electron transporting layer, the light absorbing layer, and the second electrode were formed by a general method of manufacturing the perovskite solar cell.

On the other hand, the hole transporting layer was formed by a solution casting process, not an expensive process such as deposition in the related art.

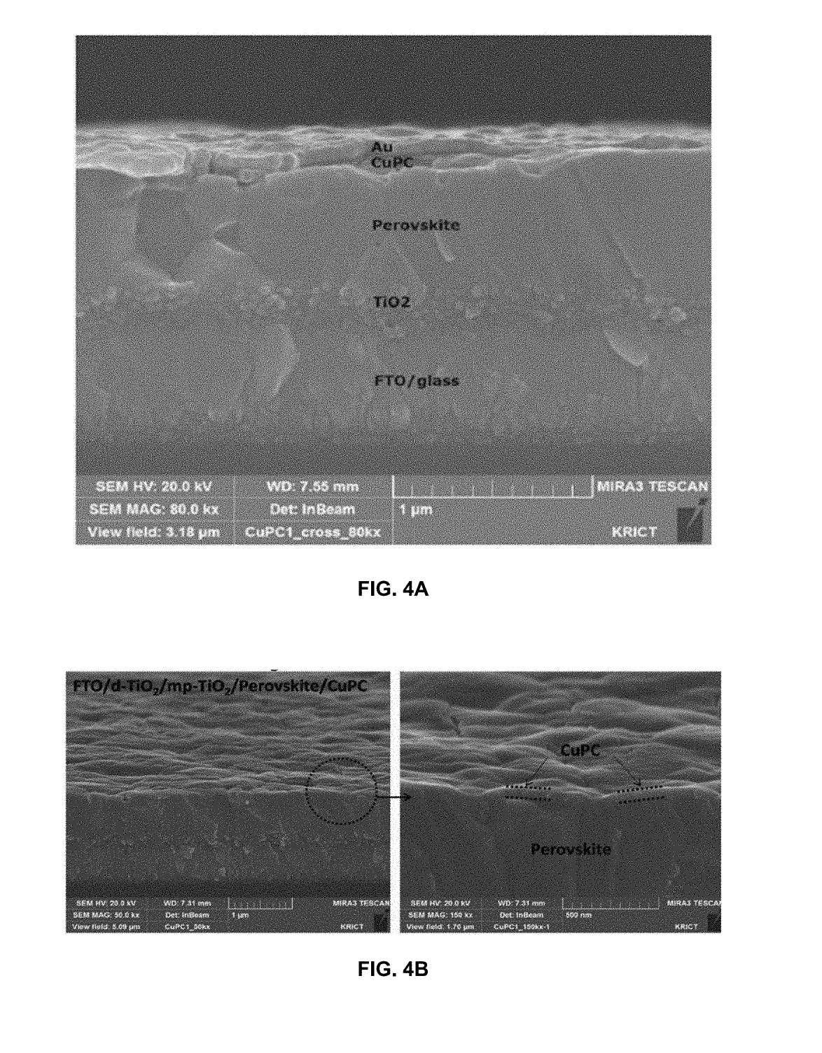

Referring to FIG. 4A, it can be seen that the perovskite solar cell has a structure in which a first electrode (FTO/Glass), an electron transporting layer (TiO₂), a light absorbing layer (Perovskite), a hole transporting layer (CuPC), and a second electrode (Au) are laminated.

### Example 3

### Evaluation of Heat Resistance of “Perovskite Solar Cell”

The heat resistance of the perovskite solar cell manufactured in Example 2 was evaluated. As Comparative Example, a perovskite solar cell in which the hole transporting layer was formed by pp-spiro, op-spiro, and PTAA was used.

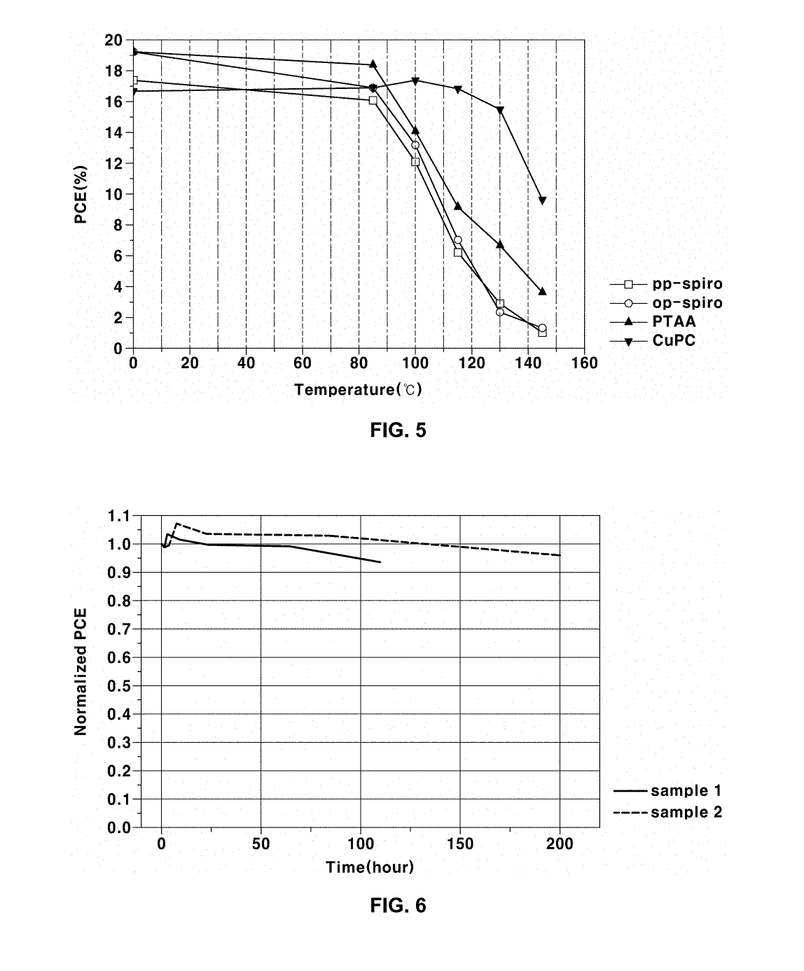

Power conversion efficiency (PCE) when each perovskite solar cell was exposed for 30 minutes at a predetermined temperature was measured. The result is illustrated in FIG. 5.

Referring to FIG. 5, it can be seen that when the temperatures of the perovskite solar cells in Comparative Example were greater than 80° C., the PCE was rapidly reduced.

On the other hand, in the perovskite solar cell in Example 2, the initial PCE was maintained at 115° C. and a reduction value of the PCE was only 5% even at 130° C.

Since embodiments of the present invention use the hole transporting material having excellent heat resistance, it can be seen that the perovskite solar cell in which the PCE is highly maintained even at a traveling environment temperature (100° C. or more) of the vehicle is provided.

### Example 4

### Evaluation of Durability of “Perovskite Solar Cell”

The durability of the perovskite solar cell manufactured in Example 2 was evaluated.

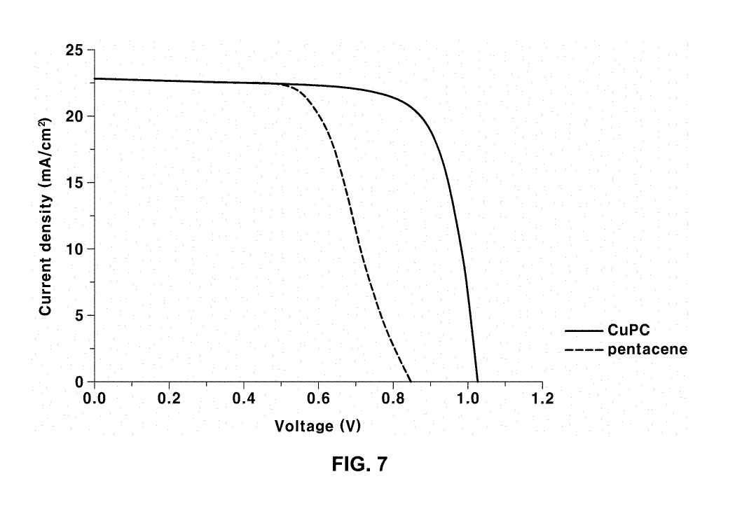

The PCE when the perovskite solar cell was left at a temperature of 85° C. and an average relative humidity of 25% to 30% for 200 hours was measured. The durability evaluation was performed twice (sample 1 and sample 2). The result is illustrated in FIG. 6. The PCE when the predetermined time elapsed was shown compared with the initial value.

Referring to FIG. 6, it can be seen that even after 200 hours elapse under the predetermined condition, the PCE at 95% or more compared with the initial value is maintained.

It can be seen that even though the perovskite solar cell according to embodiments of the present invention is exposed for a long time in a traveling environment of the vehicle, the high PCE may be stably maintained due to excellent durability.

### Example 5

### Comparison With Other Perovskite Solar Cells Including High Heat Resistant Hole Transporting Material

The perovskite solar cell was prepared by using pentacene which was an organic compound without a phase transition (thermal transition) in a temperature range of 0° C. to 300° C. like the hole transporting material according to embodiments of the present invention. Current density of the perovskite solar cell was measured and compared with current density of the perovskite solar cell in Example 2. The result is illustrated in FIG. 7.

Referring to FIG. 7, it can be seen that pentacene has heat resistance, but metal is not positioned at the center and thus efficiency and stability of the element deteriorate.

The hole transporting layer is formed by using the hole transporting material in which the phthalocyanine-based organic ligand is coordinate-bonded to the metal according to embodiments of the present invention to obtain a perovskite solar cell having PCE which is equal to or greater than PCE in the related art.

The hole transporting material according to embodiments of the present invention has excellent heat resistance to obtain a perovskite solar cell which can maintain initial PCE in a wide temperature range.

The hole transporting material according to embodiments of the present invention has excellent durability to obtain a perovskite solar cell which can maintain initial PCE even though the hole transporting material is exposed for a long time at a traveling environmental temperature of the vehicle.

When the hole transporting material according to embodiments of the present invention is used, the hole transporting layer may be easily formed by a solution casting process to be suitable for mass production, significantly reduce production costs, and ensure market competitiveness.

The invention has been described in detail with reference to embodiments thereof. However, it will be appreciated by those skilled in the art that changes may be made in these embodiments without departing from the principles and spirit of the invention, the scope of which is defined in the appended claims and their equivalents.

What is claimed is:

1. A hole transporting material for an automotive perovskite solar cell with high heat resistance in which a phthalocyanine-based organic ligand is coordinate-bonded to a metal.

2. The hole transporting material of claim 1, wherein the metal is copper (Cu), zinc (Zn) or cobalt (Co).

3. The hole transporting material of claim 1, wherein the phthalocyanine-based organic ligand includes a tert-butyl substituent.

4. The hole transporting material of claim 1, wherein the hole transporting material for the automotive perovskite solar cell having high heat resistance is expressed by Chemical Formula 1 below. 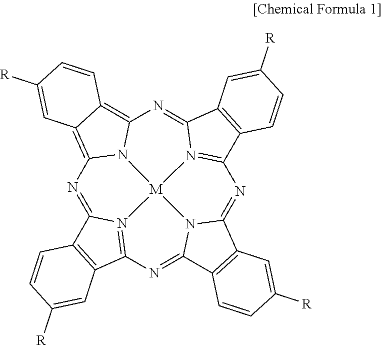
wherein, the M is copper (Cu) and the R is tert-butyl.

5. An automotive perovskite solar cell including a hole transporting layer constituted by the hole transporting material of claim 1.

6. The automotive perovskite solar cell of claim 5, comprising:
a first electrode;
an electron transporting layer formed on the first electrode;
a light absorbing layer formed on the electron transporting layer and including a compound having a perovskite structure;
a hole transporting layer formed on the light absorbing layer; and
a second electrode formed on the hole transporting layer.

7. A method for manufacturing an automotive perovskite solar cell, comprising:
forming a hole transporting layer by solution-casting the hole transporting material of claim 1.

8. The method of claim 7, wherein the solution-casting is performed by any one process selected from spin coating, spray coating, slot die, inkjet coating and gravure coating.
